(19) United States
(12) Patent Application Publication
(10) Pub. No.: US 2008/0038277 A1
(43) Pub. Date: Feb. 14, 2008

(54) Dna Pkinase Inhibitors For Treating Cancer And Diabetes

(76) Inventors: Jianhua Feng, Zurich (CH); Brian Arthur Hemmings, Bettingen (CH); Jongsun Park, Daejeon (KR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/592,874

(22) PCT Filed: Mar. 16, 2005
(86) PCT No.: PCT/EP05/02805

§ 371 Date: Jul. 16, 2007

(30) Foreign Application Priority Data
Mar. 17, 2004 (GB) 0405985.3

(51) Int. Cl. A61K 39/395; A61K 38/45; C12Q 1/48

(52) U.S. Cl. 424/158.1; 424/94.5; 435/15

(57) ABSTRACT

Methods of treating disorders associated with the PKB signalling pathway, such as cancer, diabetes, and neurodegenerative disorders, are provided. The methods comprise modulating the activity of DNA-PK; this enzyme has been identified as having serine 473 kinase activity. Compositions comprising DNA-PK or modulators thereof are also provided for use in such methods.

### FIELD OF THE INVENTION

The present invention relates to a protein kinase B (PKB) serine 473 kinase, and to methods and uses involving such a kinase. In particular, but not exclusively, the invention relates to methods and uses involving the kinase in treatment of disorders associated with the PKB signalling pathway, and in the identification of modulators of the kinase.

### BACKGROUND OF THE INVENTION

The pathway centered on protein kinase B (PKB, also called Akt) has emerged as a critical mediator of diverse cellular processes including metabolism, gene expression, migration, angiogenesis, proliferation and cell survival (1, 2). The enzyme is tightly controlled and the consequences of its deregulation have been implicated in the development of cancers and diabetes (1, 2). The activity of PKB is markedly stimulated in a phosphatidylinositol 3-kinase (PI3-kinase)-dependent manner. Upon stimulation, PKB is recruited to the plasma membrane through the binding of its N-terminal pleckstrin homology (PH) domain to phosphatidylinositol 3,4,5-trisphosphate (PIP3), a lipid product of PI3-kinase. PKB is then activated by phosphorylation on two residues: Thr-308 in the activation loop and Ser-473 in the hydrophobic motif of the C-terminal tail (3). PKB exists in three isoforms, with Thr-308 and Ser-473 referring to the residues of the PKBα isoform only. Corresponding Thr and Ser residues can be found on the β and γ isoforms, and it will be understood that reference to ‘PKB’ herein is intended to include PKBα, β, and γ, with Thr-308 and Ser-473 referring to the equivalent residues of each isoform. There is convincing evidence that Thr-308 is phosphorylated by 3-phosphoinositol-dependent kinase 1 (PDK1) (4, 5). Identification of the kinase responsible for phosphorylating Ser-473 has been a major challenge for a number of years but remains elusive. Several kinases have been reported to possess Ser-473 phosphorylating activity, including mitogen-activated protein kinase-activated kinase-2 (MAPKAPK-2) (3, 6), integrin-linked kinase (7), PDK1 (8) and PKB itself (9). However, there is evidence that these kinases are not the physiological PKB Ser-473 kinase (S473K) (3,5,10,11).

For example, activation of MAPKAPK-2 is PI 3-kinase independent, whereas PKB Ser473 phosphorylation is sensitive to PI 3-kinase inhibitors. PDK1-null cells undergo Ser473 phosphorylation, suggesting that PDK1 is not required for Ser473 phosphorylation. Furthermore, insulin-stimulated PKB Ser473 phosphorylation does not require activation of PDK1 or PKB, as Ser473 phosphorylation is not sensitive to staurosporine treatment, which inhibits PDK1 and therefore PKB activity.

Another kinase, ILK, was shown to phosphorylated glycogen synthase kinase-3, as well as Ser473 of PKB. However, it has been suggested that ILK influences PKB phosphorylation indirectly, as overexpression of certain kinase domain mutants can mimic wild type ILK in inducing Ser473 phosphorylation. Moreover, a physiological role of ILK in regulation PKB phosphorylation has been questioned since ILK knockout in Drosophila melanogaster shows a phenotype more similar to the integrin knockout than to the PKB knockout.

International patent application WO03/106669 describes a method of isolating and purifying PKB Ser473 kinase activity from cell membrane fractions. The skilled reader is referred to this publication for full details of how to identify this S473K activity. However, although this publication teaches how to obtain the S473K activity, the identity of the S473K is not determined. Thus, although the skilled person is taught how to make and use the S473K activity, identification of the molecule having S473K activity would be of benefit in providing a new target for drug discovery.

Further evidence of the difficulty in identifying the molecule having S473K activity is given by Murata et al (J Biol Chem 2003; 278 (24) 21607-14) and Hresko et al (J. Biol Chem 2003; 278 (24) 21615-22), which confirmed the work described in WO03/106669, and discuss the isolation and purification of S473K activity from cell extract. Again, however, no identification of the molecule having S473K activity is given.

The present inventors have now surprisingly identified the molecule having S473K activity as the DNA-dependent protein kinase (DNA-PK). This is a known nuclear serine/threonine protein kinase which is believed to have roles in a number of activities, including DNA double strand break repair mechanisms, and the V(D)J recombination apparatus. The known roles of DNA-PK are summarised in Smith & Jackson (1999; reference 15). Until identified by the present inventors, DNA-PK was not thought to have any role in the PKB pathway. This surprising finding provides new opportunities for drug development targets for treatment of disorders involving the PKB pathway.

### SUMMARY OF THE INVENTION

According to a first aspect of the present invention, there is provided a method of treatment of a disorder associated with the PKB signalling pathway comprising modulating the activity of a component of DNA-PK.

This method is based on the surprising identification that the DNA-PK enzyme also has S473K activity, and so would represent a target for treatment of such disorders. The disorder to be treated may be selected from cell growth anomalies, including cancer; diabetes; and neurodegenerative disorders; all associated with the PKB signalling pathway. The component of DNA-PK is preferably DNA-PKcs; although the method may comprise modulating the activity of entire DNA-PK.

‘Modulating the activity of a component of DNA-PK’ may comprise altering the interaction of DNA-PK with PKB, or may comprise altering the effective amount of DNA-PK available to the subject; for example, additional DNA-PK may be made available to the subject; or the amount of DNA-PK available to the subject may remain the same, but the interaction of the DNA-PK with PKB may be disrupted, to decrease the effective activity of the DNA-PK.

Modulation may comprise increasing or decreasing the activity of DNA-PK; this may depend on the disorder to be treated. For example, where the disorder is cancer or a neurodegenerative disorder, downregulation of DNA-PK activity may be desired; whereas upregulation of DNA-PK may be useful in treatment of diabetes.

Modulation may be accomplished by affecting the effective amount of DNA-PK available to the subject. The transcription of DNA or the translation of mRNA encoding the component of DNA-PK may be modulated; for example, by the introduction of suppressor genes or promoters into a cell expressing DNA-PK, or by the use of RNA interference (siRNA). The design and selection of suitable siRNA molecules may be accomplished by those of skill in the art. For example, a suitable siRNA has the sequence 5′-AGGGCCAAGCTGCTCACTCT-3′ (sense sequence), although alternative sequences may be used. The siRNA may be introduced into the target cell by any suitable means. Modulation of the DNA-PK activity may also be effected by providing additional DNA-PK to the subject. For example, transformed cells including additional copies of a DNA-PK encoding gene such as Prkdc may be provided to the subject; alternatively DNA-PK may be provided directly to the subject, as may nucleic acids encoding DNA-PK.

Alternatively, or in addition, activity of the DNA-PK may be modulated by directly targeting the DNA-PK enzyme. For example, PI 3 kinase inhibitors may be used to disrupt activity of DNA-PK. Suitable inhibitors may include wortmannin, quercitin, quercitrin, rutin, or LY294002. Additional inhibitors will be known to those of skill in the art. Antibodies against DNA-PK or against the Ser473 binding site on PKB may also be used to disrupt the DNA-PK activity. Compounds which competitively bind with DNA-PK in preference to PKB may also be used to modulate DNA-PK activity.

The present invention also provides the use of DNA-PK in the preparation of a medicament for treatment of a disorder associated with the PKB signalling pathway. Also provided is the use of DNA-PK in a method of treatment of a disorder associated with the PKB signalling pathway comprising modulating the activity of a component of DNA-PK. The invention also comprises a pharmaceutical composition comprising DNA-PK for use in the treatment of a disorder associated with the PKB signalling pathway.

Administration of pharmaceutical compositions of the invention may be accomplished orally or parenterally. Methods of parenteral delivery include topical, intra-arterial, intramuscular, subcutaneous, intramedullary, intrathecal, intraventricular, intravenous, intraperitoneal, or intranasal administration. In addition to the active ingredients, such compositions may comprise suitable pharmaceutically acceptable carriers comprising excipients and other components which facilitate processing of the active compounds into preparations suitable for pharmaceutical administration.

Pharmaceutical compositions for oral administration can be formulated using pharmaceutically acceptable carriers known in the art in dosages suitable for oral administration. Such carriers enable the compositions to be formulated as tablets, pills, dragees, capsules, liquids, gels, syrups, slurries, suspensions, and the like suitable for ingestion by the subject.

Pharmaceutical preparations for oral use can be obtained through combination of active compounds with a solid excipient, optionally grinding a resulting mixture, and processing the mixture of granules, after adding suitable additional compounds if desired to obtain tablets or dragee cores. Suitable excipients include carbohydrate or protein fillers such as sugars, including lactose, sucrose, mannitol, sorbitol; starch from corn, wheat, rice, potato, or other plants; cellulose such as methylcellulose, hydroxypropylmethylcellulose, or sodium carboxymethylcellulose; and gums including arabic and tragacanth; as well as proteins such as gelatin and collagen. If desired, disintegrating or solubilising agents may be added, such as cross linked polyvinyl pyrrolidone, agar, alginic acid, or a salt thereof.

Dragee cores can be provided with suitable coatings such as concentrated sugar solutions, which may also contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for product identification or to characterise the quantity of active compound.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally stabilisers. In soft capsules, the active compounds can be dissolved or suspended in suitable liquids, such as fatty oils, liquid paraffin, or liquid polyethylene glycol with or without stabilisers.

Pharmaceutical formulations for parenteral administration include aqueous solutions of active compounds. For injection, the pharmaceutical compositions of the invention may be formulated in aqueous solutions, preferably in physiologically compatible buffers such as Hanks's solution, Ringer's solution, or physiologically buffered saline. Aqueous suspension injections can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilisers or agents which increase the solubility of the compounds to allow for the preparation of highly concentrated solutions.

For topical or nasal administration, penetrants appropriate to the particular barrier to be permeated may be used in the formulation.

The pharmaceutical compositions of the present invention can be manufactured substantially in accordance with standard manufacturing procedures known in the art. According to a further aspect of the present invention, there is provided a method of screening for compounds which affect the activity of the PKB signalling pathway, the method comprising the steps of incubating a component of DNA-PK with a test compound; determining S473K activity of the DNA-PK component; and comparing S473K activity of the DNA-PK component in the presence of the test compound with such activity in the absence of the test compound. An altered activity is indicative of a compound which potentially affects the activity of the PKB signalling pathway. Such compounds may be of use in the above-described methods of treatment. Methods of determining S473K activity are described in WO03/106669, the contents of which are incorporated herein by reference; other methods may be readily determined by the skilled person, and include incubating the component of DNA-PK with PKB and assessing the phosphorylation status of Ser473 of PKB.

In one aspect of the present invention, a compound identified by the screening method may be used as a pharmaceutical. In another aspect, the compound identified by the screening method can be used for the manufacture of a medicament for the treatment of a disorder associated with the PKB signalling pathway.

PKB is a member of the AGC family of protein kinases, which share similarities in their catalytic domains including a hydrophobic motif in their C-terminus; the hydrophobic motif of most AGC kinases includes a residue corresponding to the Ser473 of PKBα. The AGC kinases are also thought to share similarities in their regulation, with full activation of the kinase dependent on phosphorylation of the hydrophobic motif. It therefore appears likely that DNA-PK may play some role in regulating AGC kinase activity.

Thus, according to a further aspect of the present invention, there is provided a method of treatment of a disorder associated with AGC kinase activity, comprising modulating the activity of a component of DNA-PK.

There is also provided a method of screening for compounds which affect the activity of an AGC kinase pathway, the method comprising the steps of incubating a component of DNA-PK with a test compound; determining serine kinase activity of the DNA-PK component; and comparing serine kinase activity of the DNA-PK component in the presence of the test compound with such activity in the absence of the test compound.

### BRIEF DESCRIPTION OF THE DRAWINGS

These and other aspects of the present invention will now be described by way of example only with reference to the accompanying drawings, which show:

FIG. 1. Identification and purification of S473K. (A-C) Mono Q fractions were assayed for S473K activity with the FSY (solid) or FAY peptides (open) in the presence (triangles) or absence (circles) of DNA. Inset in A, fractions were assayed using GST-PKBα419-480 as substrate, followed by Western blotting with pS473 phospho-specific antibody. (B) A 10-μl aliquot of each fraction was subjected to 8% SDS-PAGE and proteins visualized by Coomassie Blue staining. (C) Western blotting of fractions with anti-DNA-PKcs and anti-Ku80/Ku70 antibodies. (D) Recombinant ΔPH-PKBαT309P (PKB) was first incubated with S473K1 (5 μl), 10 μg/ml fetal calf thymus DNA, and 100 μM ATP in kinase buffer for different times. Aliquots of the reaction mixture were removed for assay of PKB activity as described in (11) or for Western blotting with pS473 and pT308 phospho-specific antibodies. (E) Fraction 27 was taken as S473K1 and kinase activity assayed with FSY peptide in the presence of DNA. Inhibitors used were LY294002 (5 μM), wortmannin (2 μM, Wort), and staurosporine (5 μM, Stauro). (F) S473K1 (fraction 27) assayed with GST-PKBα419-480 as substrate followed by Western blotting with pS473 phospho-specific antibody.

FIG. 2. DNA-PK phosphorylates and activates PKB in vitro. (A) Purified DNA-PK (Promega) (25 units) was assayed with 0.1 mg/ml of FSY, FAY and DNA-PK p53 substrate peptide (CTL) in the presence (+) or absence (−) of DNA. (B) Purified DNA-PK (25 units) was assayed using GST-PKBα419-480 as substrate followed by Western blotting with pS473 phospho-specific antibody (upper panel); the blot was then stained with Coomassie Blue (lower panel). Stauro: staurosporine (5 μM). (C) Recombinant ΔPH-PKBαT309P (PKB) was incubated with purified DNA-PK (100 units), 10 μg/ml fetal calf thymus DNA, and 100 μM ATP in kinase buffer for different times; aliquots of the reaction mixture were removed for assay of PKB activity as described in FIG. 1D or for Western blotting with pS473 and pT308 phospho-specific antibodies. (D) GST-PKBα419-480 and His-p53 (each 1 μg) were incubated with 25 units of purified DNA-PK and 100 μM [γ-32P]ATP in kinase buffer (11). The reactions were terminated and analyzed on 10% SDS-PAGE followed by autoradiography or 32P determination in excised gel slices.

FIG. 3. Phosphorylation of PKB by DNA-PK in vivo. (A) HEK293 cells were transfected with DNA-PKcs siRNA (siRNA) or control siRNA (control) for 2 days, starved for 24 h, and treated with 100 nM insulin (Ins), 0.1 mM pervanadate (Van) or left untreated (−). Aliquots (40 μg) of cell extracts were analyzed by Western blotting using phospho-specific antibodies to pS473, pT308, and total protein PKB using antibody10, and DNA-PKcs protein with monoclonal G4. (B, C) M059K, M059J, Fus1 and Fus9 cells were starved for 24 h and the cells treated with insulin (Ins) for different times before harvest. Aliquots (30 μg) of cell extracts were analyzed by Western blotting using phospho-specific antibodies pS473, pT308, and PKB protein with antibody10.

FIG. 4. Co-localization and interaction of DNA-PK with PKB at the plasma membrane. 3T3 L1 (A) and M059K cells (B) were starved for 24 h and then treated with 100 nM insulin (+Ins) for 30 min or left untreated (−Ins) before fixation. The cells were fixed in 3.7% formaldehyde, permeabilized in 2% Triton X-100 and subjected to immunostaining with anti-DNA-PKcs monoclonal G4 and antibody 10 specific for PKB. HEK293 (C) and M059K cell lysates (D) were immunoprecipitated with normal IgG (lane 1) or anti-DNA-PKcs (G4) (lane 2, 3) or anti-PKB (A4D6) (lane 4, 5) and blotted with anti DNA-PKcs (upper panel), antibody 10 (middle panel) or pS473 phospho-specific antibody (lower panel).

FIG. 5. Model of the PKB activation by upstream kinases. Following stimulation by growth factors, PI3-kinase is activated, which in turn generates the second messenger PI-3,4,5P3 to recruit PKB and PDK1 to the membrane lipid rafts, where PKB is subsequently phosphorylated on Thr-308 by PDK1, and on the hydrophobic motif Ser-473 by DNA-PK, respectively. Another member of PIKK family kinase mTOR has been purposed to be responsible for S6K hydrophobic motif phosphorylation.

FIG. 6. Analysis of S473K1 by Superdex 200 gel filtration chromatography. An aliquot (0.1 ml) of S473K1 (Mono Q fraction 27) was applied to a Superdex 200 gel filtration column equilibrated with column buffer (20 mM Tris-HCl pH 7.5, 1 mM DTT, 300 mM NaCl, and protease inhibitors). The column was eluted with the same buffer and 0.5-ml fractions were collected. S473K1 activity was determined with FSY/FAY peptides (A) or with GST-PKBα419-480 as substrate (upper panel in B). Aliquots of each fraction were immunoblotted for DNA-PKcs (lower panel in B). The elution positions of blue dextran (2,000 kDa), apoferritin (443 kDa), β-amylase (200 kDa), and bovine serum albumin (66 kDa) are indicated.

FIG. 7. Characterization of the DNA-PK recognition motif. (A) Sequence comparison of non-SQ motif for known DNA-PK substrate and hydrophobic motif for S473 kinase substrate. The original proteins of the sequence are indicated. Serine residue, phospho acceptor site, is indicated (red). (B) Purified DNA-PK together with purified Ku70/80 was incubated with each peptide derived from the AGC kinase. All reaction were performed in the presence of DNA and then subjected to p81 paper. (C) Each Ala-scanning peptide derived from PKBα was assayed with purified DNA-PK. All reaction was performed in the presence of DNA and Ku70/80. DNA-PK activity with PKBα peptide was taken as 100%. Kinase activity is the average (±SD) of three independent experiments. (D) HEK 293 cells overexpressing each PKBα mutant were treated with 0.1 uM insulin for 15 min after 18 hour serum-starvation. HA-PKBα or GST-PKBα was micro-purified from cell extracts, then PKB activity was measured with crosstide as a substrate, followed by western blot analysis with pS473 or pT308 phospho-specific antibodies and anti-PKB antibodies. Kinase activity is the average (±SD) of three independent experiments.

### DETAILED DESCRIPTION OF THE DRAWINGS

Materials and Methods

Antibodies and Chemicals

An antibody specific for PKB phosphorylated on Thr-308 (pT308) was purchased from Cell Signaling Technologies. An antibody specific for PKB phosphorylated on Ser-473 (pS473) was produced and purified as previously described (33). Polyclonal anti-PKB antibody (antibody10) (34) and monoclonal anti-PKB (A4D6) (35) have been described previously. Anti-Ku80, and anti-Ku 70 antibodies were purchased from Transduction Labs. Anti-DNA-PKcs (monoclonal G4) was from Santa Cruz Inc. Double-stranded fetal calf thymus DNA was from Sigma, and purified human DNA-PK protein and p53 substrate peptide EPPLSQEAFADLWKK (36) from Promega.

Preparation of Plasmids and Proteins

pGEX2T-PKBα419-480 was made by inserting the BamHI-EcoRI fragment of a PCR product using human PKBα cDNA as template into the pGEX2T vector (Amersham Pharmacia Biotech) and the construct verified by DNA sequencing. GST-fusion proteins were expressed in Escherichia coli BL21 strain and purified on Glutathione-Sepharose 4B (Amersham Pharmacia Biotech) according to the manufacturer's instructions. Baculovirus-expressed ΔPH-PKBβT309P was described in (37).

Purification of PKB Ser-473 Kinase

All steps were performed at 4° C. Four hundred plates (Ø 150 cm) of HEK293 cells were harvested in ice-cold phosphate-buffered saline and homogenized in ice-cold buffer A (50 mM Tris-HCl pH 7.4, 300 mM sucrose, 1 mM dithiothreitol, 10 mM sodium fluoride, 0.1 mM sodium orthovanadate, 20 nM okadiac acid, 1 mM phenylmethanesulfonyl fluoride and 1 mM benzamidine) using a polytron homogenizer. The nuclear fraction was removed by centrifuging at 1000 g for 10 min and the resulting supernatant was further centrifuged at 100,000 g for 60 min. The crude membrane pellets were suspended in buffer B (20 mM Tris-HCl pH 7.4, 0.5 M NaCl, 1 mM dithiothreitol, 1 mM phenylmethanesulfonyl fluoride and 1 mM benzamidine). Following a 30-min incubation at 4° C., insoluble proteins were pelleted by centrifugation at 100,000 g for 20 min and the supernatant was dialyzed against buffer C (20 mM Tris-HCl pH 7.4, 1 mM EDTA, 1 mM EGTA, 1 mM dithiothreitol, 1 mM phenylmethanesulfonyl fluoride, 1 mM benzamidine, and 5% glycerol) for 2 h. After a brief centrifugation, the supernatant was loaded on to a Q-Sepharose Fast Flow column (2.6×8 cm) equilibrated with buffer C. The column was then extensively washed with buffer C plus 0.1 M NaCl and developed with a continuous gradient of NaCl (0.1-0.6 M) in buffer C. The fractions containing S473K activity were pooled and dialyzed against buffer C for 2 h prior to loading onto a Mono S(HR10/10) column. After washing with 10 volumes of buffer C, proteins were eluted with a continuous NaCl gradient (0-0.6 M) in buffer C. The kinase activity eluted between 0.34 M-0.38 M NaCl. Fractions were pooled and dialyzed against buffer C. The dialyzed samples were applied onto a Mono Q (HR5/5) column. The column was washed with 10 volumes of buffer C and the proteins were eluted in a continuous NaCl gradient (0-0.5 M) in buffer C.

Gel Filtration Analysis

Gel filtration was performed with a Superdex-200 HR10/30 column attached to an FPLC system (Amersham Pharmacia Biotech) as described previously (38).

Protein Kinase Assay

S473K activity was assayed using the peptide RRPHFPQFSYSASSTA corresponding to the C-terminus of PKBα (FSY peptide). A second peptide in which Ser-473 was changed to alanine was used to account for background and phosphorylation on other residues (FAY peptide) as described previously (33). In parallel, the S473K activity was also monitored by an alternative kinase assay method using a C-terminus of GST-PKBα as substrate followed by Western blotting with a Ser-473 phospho-specific antibody. Briefly, assays were performed in 30-μl reactions containing 30 mM Tris-HCl pH 7.4, 1 mM DTT, 10 MgCl2, 1 μM PKI, 1 μM microcystin-LR, 150 μM ATP, 1 μg GST-PKBα419-480 and enzyme. After incubating for 30 min at 30° C., reactions were stopped by adding SDS sample buffer, boiled for 3 min at 95° C., and then resolved by SDS-PAGE followed by Western blotting with phospho-specific Ser-473 antibody. The in vitro PKB kinase assay was as described previously using the specific peptide RPRAATF (R7Ftide) as substrate (39).

Cell Culture and Transfections

HEK293 and 3T3L1 cells were grown in DMEM supplemented with 10% fetal calf serum, 100 units/ml of penicillin, and 100 μg/ml of streptomycin. Transfections of PKB constructs were described previously (34). For RNA interference, HEK293 cells were transfected with the DNA-PKcs siRNA or a 21-nt irrelevant RNA duplex as a control using oligofectamine (Invitrogen). The targeted sequence of human DNA-PKcs siRNA selected was 5′-AGGGCCAAGCTGCTCACTCT-3′ (sense sequence), and the 21-nt synthetic siRNA duplex was prepared by Qiagen. M059J and M059K were from ATCC and grown in DMEM supplemented with 10% foetal calf serum, 1% non-essential amino acids, 4 mg/ml glutamine, 100 units/ml penicillin, and 100 μg/ml streptomycin. M059J/Fus1 and M059J/Fus9 cells were grown in the same medium containing 250 μg/ml Geneticin. For analytical experiments, cells were starved overnight prior to treatment and the cells were lysed in NP-40 lysis buffer (34).

Immunofluorescence

For cell immunofluorescence, the cells were fixed with 3.7% formaldehyde and permeabilized with 0.2% Triton-X 100. After blocking, cells were incubated with monoclonal anti-DNA-PK antibody (G4, 2 μg/ml diluted in PBS) for 2 h, followed by polyclonal anti-PKB antibody (antibody10, 2 μg/ml diluted in PBS) for a further 2 h. Cells were then extensively washed and incubated with FITC-conjugated anti-mouse IgG and TR-conjugated anti-rabbit IgG (both 1:100 dilution). After washing, the cells were visualized by confocal microscopy.

Immunoprecipitation

Extracts (500 μg) were diluted in co-immunoprecipitation buffer (20 mM Tris-HCl pH 7.5, 150 mM NaCl, 1 mM benzamidine, 1 mM phenylmethanesulfonyl fluoride, 500 μl) with monoclonal DNA-PK (G4, 2 μg) or monoclonal PKB (A4D6, 2 μg) antibodies and protein G beads (20 μl, Amersham). After 6 h continuous gentle agitation at 4° C., the beads were collected by pulse spin and washed 3 times in co-immunoprecipitation buffer, after which they were resuspended in SDS sample buffer and heated at 95° C. for 3 min before analysis by SDS-PAGE and Western blotting.

Experimental Data

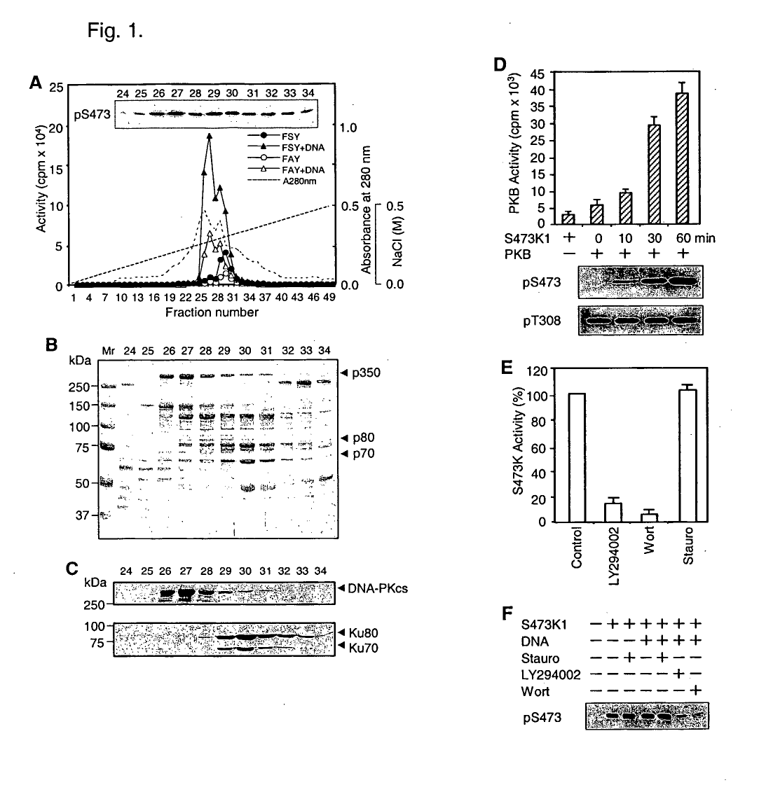

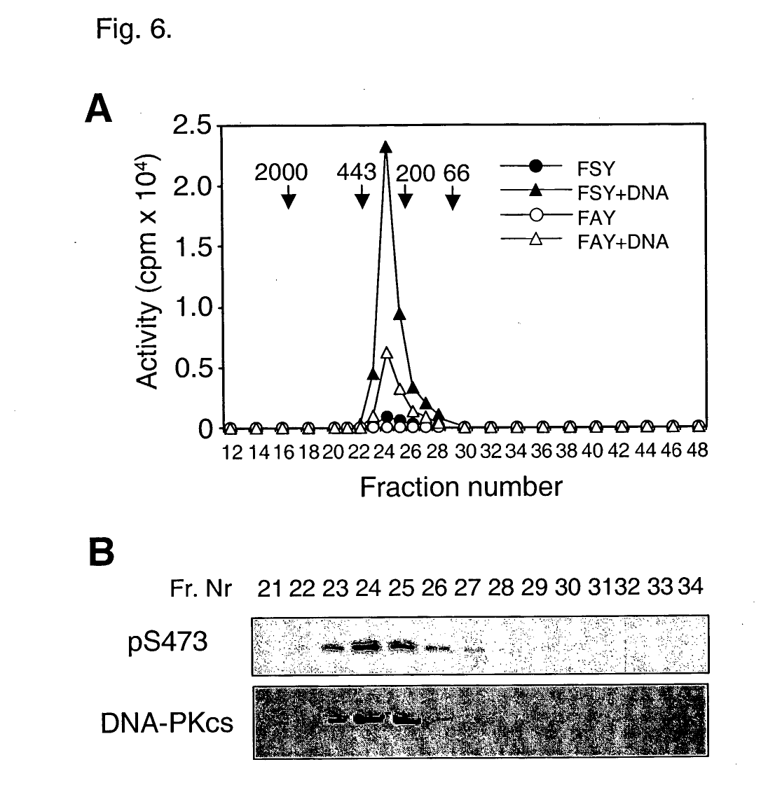

We previously identified a S473K activity in lipid rafts of plasma membrane (11) and now report an efficient method for purification and identification of this kinase from HEK293 cells (12). The purification procedure was monitored using a peptide derived from the hydrophobic motif of PKBα containing Ser-473 (FSY) and a mutant peptide with Ser-473 mutated to Ala (FAY) as substrates (12-13). In parallel, specific S473K activity was also monitored using GST-PKBα419-480 as substrate, followed by Western blotting with a Ser-473 phospho-specific antibody (13). This combined assay approach allowed the identification of the enzymes activity specifically targeting Ser-473 of PKB. The procedures for purification of S473K, including membrane fractionation, chromatography on Q-Sepharose, Mono S, and Mono Q columns are described in (13). The elution profile from Mono Q (FIG. 1) shows two distinct kinase activities. S473K activity in fraction 27 (S473K1) specifically phosphorylated recombinant GST-PKBα419-480, with only poor activity towards the peptides (FIG. 1A). The second activity (S473K2) peaking at fraction 30 phosphorylated both recombinant PKBα419-480 and Ser-473 peptides (FIG. 1A). SDS-PAGE analysis of Mono Q fractions revealed one major band of apparent molecular mass approximately 350-kDa whose elution profile paralleled S473K1 activity (FIGS. 1A and B). Furthermore, the 350-kDa band co-eluted with S473K1 activity on gel-filtration chromatography (FIG. 6). The band corresponding to the 350-kDa protein was analyzed by mass-spectrometry and the sequences of 65 peptides obtained all showed a perfect match to the human DNA-dependent protein kinase catalytic subunit (DNA-PKcs) (14). Western blot analysis of Mono Q fractions revealed that the 350-kDa bands corresponded to DNA-PKcs, which exactly paralleled S473K1 activity (FIG. 1C). DNA-PKcs is a member of the large phosphatidylinositol 3-kinase-related kinase (PIKK) family that includes mTOR (mammalian target of rapamycin), the ataxia telangiectasia gene product (ATM), and ATM- and RAD-3-related kinase (ATR) (15). The holoenzyme of DNA-PK consists of a 350-kDa catalytic subunit (DNA-PKcs) and a Ku antigen complex Ku80/Ku70 (15-17) that has been reported to be required for V(D)J recombination, DNA repair, and transcriptional regulation (see review 15). Interestingly, the peaks of two other DNA-PK components, Ku80/Ku70, were partially resolved from the DNA-PKcs peak (FIG. 1C). This suggests that the partially purified DNA-PKcs can significantly phosphorylate PKB in the absence of Ku80/Ku70 subunits. Gel-filtration analysis of Mono Q fraction 27 (S473K1), S47K1 activity eluted at approximately 350-kDa and closely paralleled DNA-PKcs (FIG. 6). These results suggest that DNA-PKcs is not always complexed with the Ku80/Ku70 subunit, consistent with several earlier observations (15-17). As DNA-PK is known to be activated by DNA ends (15), we tested the DNA dependence of S473K1 by assaying the kinase activity of Mono Q fractions in the presence of linear double-stranded fetal calf thymus DNA. As expected, the S473K1 activity towards the substrate FSY peptide was stimulated greatly by DNA and peaked at fraction 27, corresponding exactly to DNA-PKcs (FIG. 1A-C). It is worth noting that DNA also stimulates S473K2 activity probably due to contamination of S473K2 fractions with DNA-PKcs (FIGS. 1A and B).

To test whether Ser-473 phosphorylation catalyzed by purified S473K1 is sufficient for activation of PKB activity, a monophosphorylated form of recombinant PKB (ΔPH-PKBβT309P prepared by in vitro phosphorylation with PDK1 [18]) was first phosphorylated by S473K1 and PKB assayed using RPRAATF (R7Ftide) as substrate (13). As shown in FIG. 1D, ΔPH-PKBβT309P activity towards R7Ftide was dramatically increased to approximately 10-fold after incubation with S473K1. The Ser-474 phosphorylation of ΔPH-PKBβT309P was also increased in a time-dependent manner (FIG. 1D). To determine whether DNA-PK is a major activity in the S473K1 fraction, we further characterized this fraction using PI3-kinase inhibitors known to be effective inhibitors of DNA-PK activity (15, 16). As shown in FIGS. 1E and F, S473K1 was potently inhibited by LY294002 and wortmannin indicating that DNA-PKcs is a major active component of S473K1. This result is consistent with previous cell culture data that showed Ser-473 phosphorylation is sensitive to these two inhibitors (3). Our previous studies revealed that S473K is staurosporine insensitive (11). Consistent with this, S473K1 activity was not affected by 5 μM staurosporine (FIGS. 1E and F). Based on these results, we suggest that DNA-PKcs is the major activity corresponding to the physiological S473K. In contrast, S473K2 fraction is sensitive to staurosporine (14), suggesting that S473K2 is distinct from S473K1.

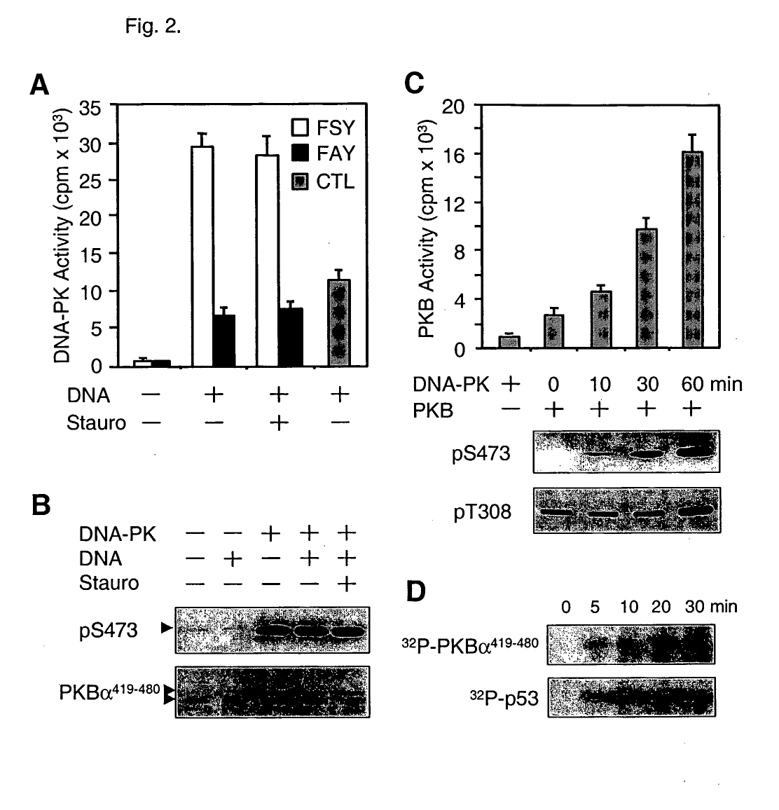

To confirm that DNA-PKcs is an active component of S473K, purified DNA-PK (from HeLa cells and human placenta) was tested for their ability to phosphorylate PKB on Ser-473. As shown in FIGS. 2A and B, both the FSY peptide and GST-PKBα419-480 were efficiently phosphorylated by purified DNA-PK in the presence of DNA. Strikingly, DNA-PK significantly phosphorylated GST-PKBα419-480 but not the FSY peptide in the absence of DNA (FIG. 2B) and this result is consistent with S473K1 activity (FIG. 1A). In addition, the phosphorylation of PKB catalyzed by purified DNA-PK was inhibited by LY294002 and wortmannin (15) but was resistant to staurosporine (FIGS. 2A and B). Next, we tested the phosphorylation of ΔPH-PKBβT309P by purified DNA-PK under the conditions described above. As shown in FIG. 2C, PKB activity towards R7Ftide was enhanced more than eight-fold with increasing Ser-473 phosphorylation in a time-dependent manner, similar to that observed with our partially purified S473K1 fraction (FIG. 1D). The stoichiometry of GST-PKBα419-480 phosphorylation was ˜0.3 mol phosphate/mol protein, comparable to phosphorylation of p53 by DNA-PK (FIG. 2D). Thus, the properties of the purified DNA-PK coincide perfectly with the properties of our partially purified S473K1 activity, further supporting the concept that DNA-PK is a S473K.

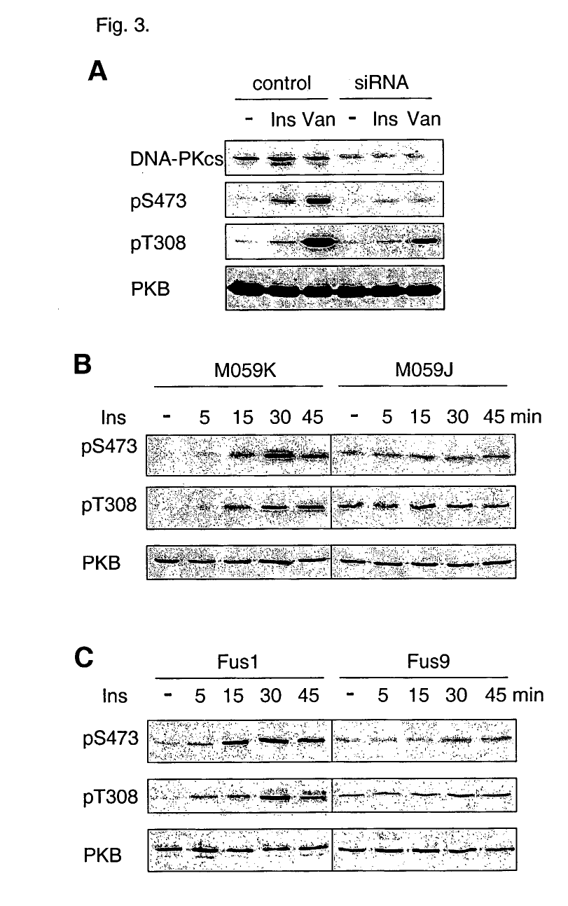

To confirm that the DNA-PK can function as a S473K in vivo we transfected HEK293 cells with specific small interfering RNA (siRNA) leading to a marked loss of DNA-PKcs protein (FIG. 3A, upper panel). Stimulation of siRNA transfected cells with insulin or pervanadate markedly reduced PKB phosphorylation on Ser-473 phosphorylation but did not markedly affect Thr-308 phosphorylation (FIG. 3A). Thus, DNA-PK again appears to be required for Ser-473 phosphorylation. The human glioblastoma cell line M059J lacks DNA-PKcs and DNA-PK activity, while the related cell line M059K contains wild-type DNA-PK (19) Treatment of cells with insulin increased phosphorylation of both Ser-473 and Thr-308 residues in M059K cells but there was no significant increase in M059J cells (FIG. 3B). Surprisingly the level of phosphorylation of both sites was higher in starved M059J cells suggesting further alterations in PKB regulation in this transformed cell. To further determine whether the insulin-resistant phenotype of PKB phosphorylation is due to a defect in DNA-PK in M059J cells, rescue experiments were performed with two further cell lines, M059J/Fus1 cells generated from M059J by cell fusion leading to DNA-PK expression, and M059J/Fus9 control cells that lack DNA-PKcs (20). The results in FIG. 3C clearly show that M059J/Fus1 cells regained an insulin-sensitive phenotype for Ser-473 phosphorylation similar to that of M059K, whilst the control M059J/Fus9 cells showed extremely low Ser-473 phosphorylation. Thus Ser-473 phosphorylation can be restored by complementation of DNA-PKcs-defective cells with the Prkdc gene, again implicating DNA-PK as a determinant kinase responsible for Ser-473 phosphorylation. Interestingly, Thr-308 phosphorylation was also reduced in Fus9 cells. This persistent but low Ser-473 phosphorylation in DNA-PKcs-deficient cells (FIGS. 3 A; B and C) indicates the existence of additional kinase(s) required for Ser-473 phosphorylation. This could correspond to the S473K2 activity identified in the MonoQ fraction (FIG. 1A). Additionally, the status of Ser-473 phosphorylation of PKB in DNA-PK knockout mouse embryonic fibroblast (MEF) cells was monitored [41]. DNA-PK knockout MEFs didn't properly respond to insulin stimulation in Ser-473 phosphorylation of PKB whereas wild type control MEFs show a nice increase in Ser-473 phosphorylation in a time-dependent manner. In order to confirm that DNA-PK is responsible for Ser-473 phosphorylation in these MEFs, DNA-PK was immunoprecipitated with anti-DNA-PK antibodies (G-4) from each MEFs, then incubated with FSYtide, FAYtide [42] or p53tide [43] in vitro. DNA-stimulated DNA-PK activity toward each peptide was only observed in wild type MEFs not in DNA-PK knockout MEFs, suggesting that DNA-PK is indeed responsible kinase to phosphorylate FSYtide and p53tide in DNA-dependent manner. Furthermore similar results were obtained from DNA-PK deficient cells.

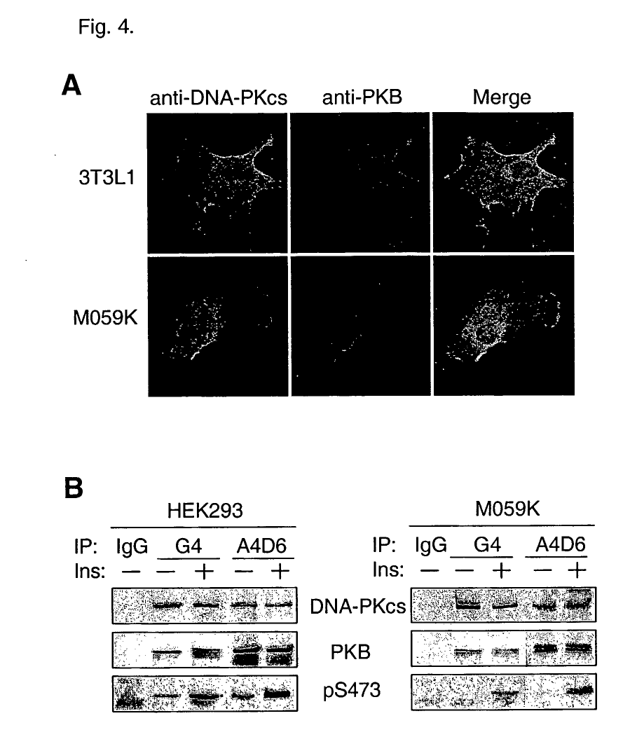

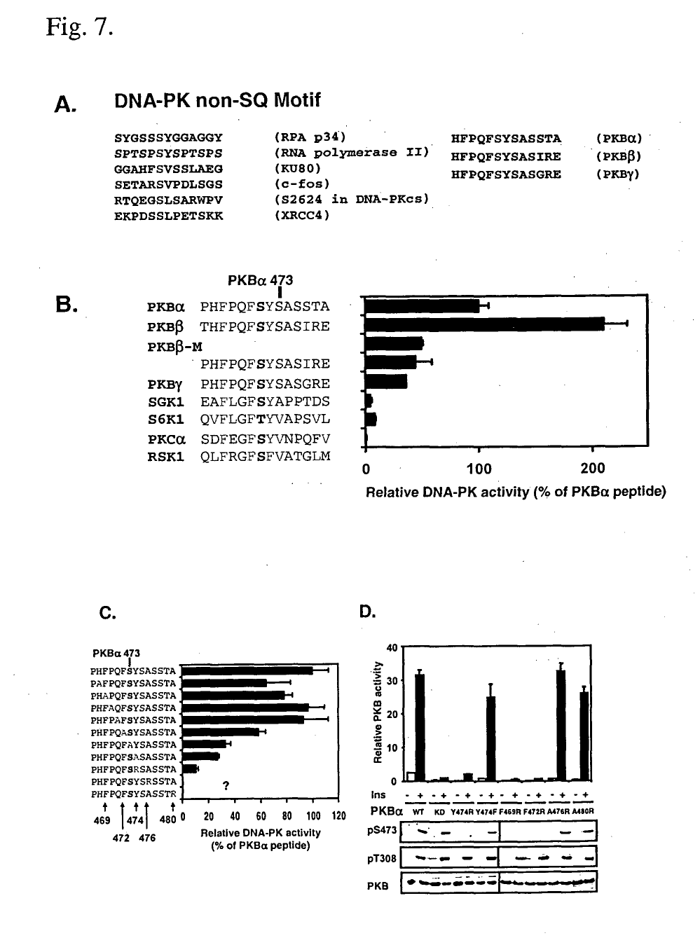

It has been shown that efficient DNA repair requires growth factor signaling (21) and that this effect may be due to the physical association of DNA-PK with epidermal growth factor receptor (EGFR) (22). Significantly inositol hexakisphosphate (IP6) was reported to bind to DNA-PK (23), suggested that IP6 might play pivotal roles in modulating the localization and/or biological properties of DNA-PK. More recent data indicate a novel localization of the DNA-PK complex in lipid rafts (24) in line with our finding that PKB S473K is associated with plasma membrane lipid rafts (12). Subcellular fractionation of HEK293 cells revealed substantial DNA-PKcs in the membrane fraction (14). We tested whether DNA-PKcs and PKB colocalized in M059K, HEK293 and 3T3L1 cells (FIGS. 4A and B). DNA-PKcs was present mainly in the nucleus, but with a substantial amount also in the cytosol and at the membrane, whereas PKB was mainly cytosolic with a fraction associated with the membrane in 3T3L1 and M059K cells. Significantly DNA-PK was co-localized with PKB at the plasma membrane in these cells. To determine whether DNA-PKcs associates with PKB, HEK293 and M059K cells were starved and treated with insulin for 30 min. Cells were lysed in NP-40 lysis buffer and immunoprecipitated with DNA-PK or PKB specific antibodies (FIG. 4B). The results reveal that PKB is associated with DNA-PKcs and following insulin treatment is phosphorylated on Ser-473. PKB belongs to the AGC family of protein kinases that possess a highly conserved T-loop in the central kinase domain and a hydrophobic motif in the C-terminus (see review 25). The hydrophobic motif of most AGC kinases is characterized by a conserved motif: F-x-x-F-S/T-Y/F (the S/T residue is equivalent to Ser473 of PKBα) (25). Significantly mTOR, a close relative of DNA-PK in the PIKK family, phosphorylates p70S6 kinase on Thr-389 of hydrophobic motif (26). DNA-PK phosphorylates many protein substrates on Ser/Thr residues followed by glutamine, i.e. the “S/TQ” motif (27, 28). However, DNA-PK also phosphorylates proteins at so-called “non-S/TQ” sites, with a preference for Ser/Thr residues followed by a hydrophobic amino acid (15, 27, 28). It is noteworthy that DNA-PK appears to have a predisposition for phosphorylation sites at the extreme termini of its substrates; this may indicate better accessibility of the substrate and the active site of the large kinase complex (28). The substrate specificity of DNA-PK warrants further investigation, our initial data indicate the PKB FSY peptide is about 6 times more effective than the p53 peptide (14). We also tested DNA-PK with several other hydrophobic motif peptides (RSK1, RSK3, PKCα, NDR2, S6K-1 and SGK-1) modeled on the PKB site, and found that only those from the three PKB isoforms served as substrate (14). Additionally, to characterize further the substrate specificity of DNA-PK for hydrophobic motif substrate, we further test the hydrophobic motif peptide from other AGC kinases. Strikingly, the peptides derived from PKB isoform are preferentially phosphorylated by DNA-PK. Among the peptide of PKB isoforms, PKBβ peptide exhibits the highest activity. However, this activity is due to additional Thr in N-terminal of peptide since the activity of mutant peptide (PKBβ-M) is dramatically reduced similar to PKBγ peptide. These results also confirmed that the hydrophobic motif of PKB is specifically phosphorylated by DNA-PK. To study the influence of amino acid residues adjacent to the minimal consensus phosphorylation site for DNA-PK (FxxFSY) on the recognition of substrates, the inventors generated variant Ala-scanning peptide. By comparing the phosphorylation of FSYtide with the variant Ala-scanning peptides, it is clear that phenyalanine at position +1 and +4 and tyrosine at position −1 from phospho-acceptor site are required for DNA-PK activity in the FSYtide since the mutant peptide (F474R, FIG. 7) which was substituted hydrophobic amino acid to basic amino acid, do not show any activity. The inventors further validated these observations at the protein-level by using the over-expression of mutant protein in cell culture system. As shown in the FIG. 7D, insulin-mediated PKB activation is impaired in F469R and F472R mutant which is substituted hydrophobic amino acid to basic amino acid in HEK 293 cells (FIG. 7C).

Mouse and human cells deficient in DNA-PK are hypersensitive to ionizing radiation and to radiomimetic drugs (15); a similar phenotype can be observed in Akt1/PKB knockout mice (29). DNA-PK is activated upon DNA damage by UV-irradiation, as is PKB (30). Induction of apoptosis by cisplatin was explained by a decrease in DNA-PK activity through proteolytic degradation of DNA-PK (31); however, PKB activity and Ser-473 phosphorylation were also inhibited by cisplatin treatment (32).

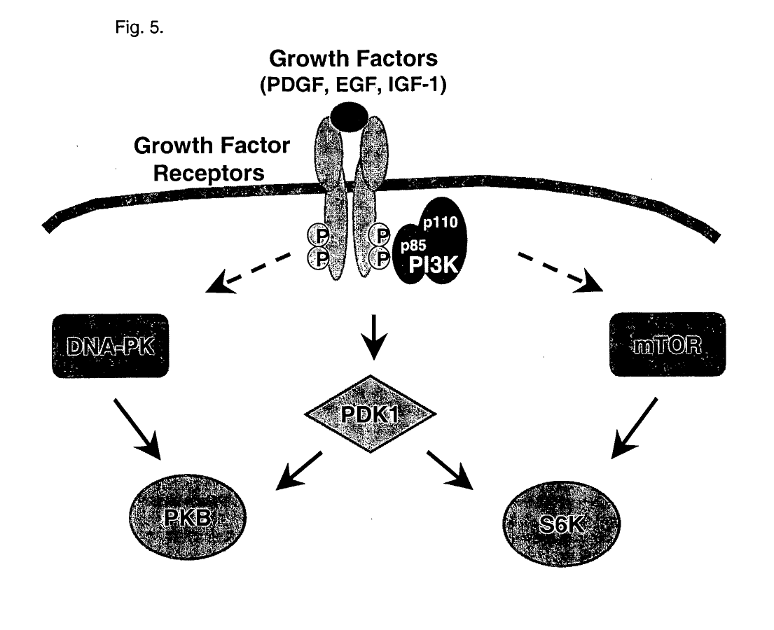

The identification of DNA-PK as the elusive Ser-473 hydrophobic motif kinase is unexpected and several important questions are posed by this discovery. DNA-PK pathway plays a crucial role in controlling transcription, the cell cycle progression, and apoptosis (15). Similarly PKB is also implicated in the regulation of many different cellular processes. It is now important to integrate these two fields and to understand precisely how the PI3Kinase pathway regulates DNA-PK. Our current view of how PKB is regulated by PDK1/DNA-PK is shown in FIG. 5, and also shows the parallels with p70S6K regulation by PDK1/mTOR. This leads to the suggestion that maybe other members of the PIKK family could function as hydrophobic motif kinases.

### REFERENCES

1. S. R. Datta, A. Brunet, M. E. Greenberg, Genes & Dev. 13, 2905 (1999).
2. D. P. Brazil and B. A. Hemmings, Trends Biochem. Sci. 26, 657 (2001).
3. D. R. Alessi et al., EMBO J. 15, 6541 (1996).
4. D. R. Alessi et al., Curr. Biol. 7, 261 (1997).
5. M. R. Williams et al., Curr. Biol. 10, 439 (2000).
6. M. J. Rane et al., J. Biol. Chem. 276, 3517 (2001).
7. M. Delcommenne et al., Proc. Natl. Acad. Sci. USA 95, 11211 (1998).
8. A. Balendran et al., Curr. Biol. 9. 393 (1999).
9. A. Toker and A. C. Newton, J. Biol. Chem. 25, 8271 (2000).
10. C. G. Zervas, S. L. Gregory, N. H. Brown, J. Cell Biol. 152, 1007 (2001).
11. M. M. Hill et al., J. Biol. Chem. 276, 25643 (2001).
12. M. M. Hill, J. Feng, B. A. Hemmings, Curr. Biol. 12, 1251 (2002).
13. Materials and Methods are described above.
14. J. Feng, J. Park, D. Hess, B. A. Hemmings, data not shown.
15. G. C. M. Smith and S. P. Jackson, Genes & Dev. 13, 916 (1999).
16. S. P., Lees-Miller, Y. R. Chen, C. W. Anderson, Mol. Cell Biol. 10, 6472 (1990).
17. A. Dvir, L. Y. Srein, B. L. Calore, W. S. Dynan, J. Biol. Chem. 268, 10440 (1993).
18. J. Yang et al., Mol. Cell 9, 1227 (2002).
19. M. J. Allalunis-Turner, G. M. Barron, R. S. 3rd. Day, K. D. Dobler, R. Mirzayans, Radiat. Res. 134, 349 (1993).
20. B. S. Hoppe, R. B. Jensen, C. U. Kirchgessner, Radiat. Res. 153, 125 (2000).
21. D. Bandyopadhyay, M. Mandal, L. Adam, J. Mendelsohn, R. Kumar, J. Biol. Chem. 273, 1568 (1998).
22. L. A. Hanakahi, M. Bartlet-Jones, C. Chappell, D. Pappin, S. C. West, Cell 102, 721 (2000).
23. J. Mendelsohn and Z. Fan, J. Natl. Cancer Inst. 89, 341 (1997).
24. H. Lucero, D. Gae, G. E. Taccioli, J. Biol. Chem. 278, 22136 (2003).
25. N. R. Leslie, R. M. Biondi, D. R. Alessi, Chem. Rev., 101, 2365 (2001).
26. P. E. Burnett, R. K. Barrow, N. A. Cohen, S. H. Snyder, D. M. Sabatini, Proc. Natl. Acad. Sci. USA 95, 1432 (1998).
27. M. Zernik-Kobak, K. Vasunia, M. Connelly, C. W. Anderson, K. Dixon, J. Biol. Chem. 272, 23896 (1997).
28. D. W. Chan, R. Ye, C. J. Veillette, S. P. Lees-Miller, Biochemistry 38, 1819 (1999).
29. W. S. Chen et al., Genes & Dev. 15, 2203 (2001).
30. C. Huang et al., J. Biol. Chem. 276, 40234 (2001).
31. K. M. Henkels and J. J. Turchi, Cancer Res. 57, 4488 (1997).
32. E. Piccolo et al., Oncogene 23, 1754 (2004).
33. M. M. Hill and B. A. Hemmings, Methods Enzymol. 345, 448 (2002).
34. M. Andjelkovic et al., J. Biol. Chem. 272, 31515 (1997).
35. S. M. Maira et al., Science 294, 374 (2001).
36. S. P. Lees-Miller, et al., Mol. Cell. Biol. 12, 5041 (1992).
37. J. Yang et al., Mol. Cell 9, 1227 (2002).
38. M. M. Hill, J. Feng, B. A. Hemmings, Curr. Biol. 12, 1251 (2002).
39. D. R Alessi, F. B. Caudwell, M. Andjelkovic, B. A. Hemmings, P. Cohen, FEBS Lett. 99, 333 (1996).
40. J. Feng, J. Park, P. Cron, D. Hess, B. A. Hemmings, J. Biol. Chem. 279, 41189 (2004).
41. G. E. Taccioli et al., Immunity 9, 355 (1998).
42. R. A. Woo, M. T. Jack, Y. Xu, S. Burma, D. J. Chen, P. W. Lee, EMBO J. 21, 3000 (2002).
43. S. P. Lees-Miller, K. Sakaguchi, S. J. Ullrich, E. Appella, C. W. Anderson, Mol. Cell Biol. 12, 5041 (1992).

We claim:

1. A method of treatment of a disorder associated with the PKB signaling pathway comprising modulating the activity of a component of DNA-PK.

2. The method of claim 1, wherein the disorder is selected from cell growth anomalies, including cancer; diabetes; and neurodegenerative disorders.

3. The method of claim 1, wherein the component of DNA-PKis I DNA-PKcs.

4. The method of claim 1, wherein the step of modulating the activity of a component of DNA-PK comprises altering the interaction of DNA-PK with PKB.

5. The method of claim 4, wherein the interaction of DNA-PK with PKB is modulated by means of a PI 3 kinase inhibitor.

6. The method of claim 4, wherein the interaction of DNA-PK with PKB is modulated by means of an antibody.

7. The method of claim 1, wherein the step of modulating the activity of a component of DNA-PK comprises altering the effective amount of DNA-PK available to the subject.

8. The method of claim 7, wherein the activity of the component of DNA-PK is modulated by means of RNA interference.

9. The method of claim 7, wherein the activity of the component of DNA-PK is modulated by means of providing additional DNA-PK to the subject.

10. The use of a component of DNA-PK in the preparation of a medicament for treatment of a disorder associated with the PKB signaling pathway.

11. The use of a component of DNA-PK in a method of treatment of a disorder associated with the PKB signaling pathway comprising modulating the activity of a component of DNA-PK.

12. A pharmaceutical composition comprising a component of DNA-PK for use in the treatment of a disorder associated with the PK13 signaling pathway.

13. A method of screening for compounds which affect the activity of the PKB signaling pathway, the method comprising the steps of incubating a component of DNA-PK with a test compound; determining S473K activity of the DNA-PK component; and comparing S473K activity of the DNA-PK component in the presence of the test compound with such activity in the absence of the test compound.

14. A method of treatment of a disorder associated with AGO kinase activity, comprising modulating the activity of a component of DNA-PK.

15. A method of screening for compounds which affect the activity of an AGC kinase pathway, the method comprising the steps of incubating a component of DNA-PK with a test compound; determining serine kinase activity of the DNA-PK component; and comparing serine kinase activity of the DNA-PK component in the presence of the test compound with such activity in the absence of the test compound.
